(19) United States
(12) Patent Application Publication
(10) Pub. No.: US 2011/0206732 A1
(43) Pub. Date: Aug. 25, 2011

(54) ALBUMIN FOR TREATING CANCER AND A PHARMACEUTICAL COMPOSITION COMPRISING THE COMBINATION OF AN ANTI-NEOPLASTIC AGENT AND ALBUMIN

(76) Inventors: Giampiero Pescarmona, Torino (IT); Antonio Ponzetto, Moncalieri (Torino) (IT); Gianfranco Merizzi, Torino (IT); Luisa Gennero, Torino (IT)

(21) Appl. No.: 12/673,750

(22) PCT Filed: Aug. 19, 2008
(86) PCT No.: PCT/IB08/53316

§ 371 Date: Feb. 16, 2010

(30) Foreign Application Priority Data
Aug. 21, 2007 (IT) TO2007A000608

(51) Int. Cl. A61K 39/04; A61K 38/38; A61K 38/12; A61P 35/00

(52) U.S. Cl. 424/248.1; 514/19.3; 514/19.9

(57) ABSTRACT

The present invention relates to albumin as an active ingredient in a medicament for the treatment of cancer and to a pharmaceutical composition comprising at least one anti-neoplastic agent and albumin at a concentration of at least 2% w/v of the composition, in a pharmaceutically acceptable vehicle. The anti-neoplastic agent is preferably hydroxyurea, preferably at concentration between 10 μM and 1 mM, more preferably between 10 μM and 100 μM. The pharmaceutical composition of the invention is effective in the therapeutic treatment of cancer and advantageously shows reduced side effects in comparison with the administration of the anti-neoplastic agent alone.

The present invention relates to a novel therapeutic application of albumin and a novel pharmaceutical composition for the treatment of cancer.

Many anti-neoplastic agents are known for use in the treatment of cancer, among which there is the cytostatic agent hydroxyurea (HU), also known as hydroxycarbamide. Hydroxyurea lowers the intracellular concentration of endogenous nucleotides that are required for DNA replication, by inhibiting the enzyme ribonucleotide reductase [1-2].

Significant therapeutic responses to hydroxyurea treatment are obtained in chronic myeloid leukaemia and in the other chronic myeloproliferative syndromes (essential thrombocytemia, polycytemia vera and idiopathic myelofibrosis) with a continuous therapy at the dose of 20-30 mg/Kg/day per os in one or two daily administrations for six weeks of treatment [1-2]. Hydroxyurea, at the same therapeutic dosages, is also recommended in the treatment of subjects suffering from homozygous sickle-cell anaemia and it exhibits potential action as a co-adjuvant in the anti-retroviral therapy [1].

Hydroxyurea is a cytostatic agent that easily penetrates several tissues, such as the lymph nodes and brain. The main side effect in case of HU therapy is the medullary depression, which causes leukopaenia, anaemia and sometimes thrombocytopaenia (reduction in the white blood cell, red blood cell and platelet count) due to the inhibition of cell replication [1-2]. Medullary depression becomes manifest with anaemia, and consequent tiredness; tendency to develop ecchymoses, that is bruises or haemorrhages; enhanced risk of developing infections. This effect is generally mild. The reduction in the number of haematic cells can start to appear approximately 7 days after drug administration and usually the haematic cell count reaches the lowest values at 10-14 days after chemotherapy. The haematic cell count then starts to rise again constantly, and usually returns to normal by 21-28 days. The haematic cell count decreases as a function of the dose of hydroxyurea. Should a decided reduction in the haematic cell count occur, it is advisable to suspend the administration of the drug so as to allow for the recovery of the bone marrow functionalities [2].

Moreover, at the common doses used in oncologic therapy, hydroxyurea may cause skin rashes, that is a skin eruption, similar to acne, which can produce itchiness, dryness or pain in the oral cavity, vomiting or nausea, and infertility [3].

These effects are evident at the doses used in oncology.

The object of the present invention is to provide a medicament having anti-neoplastic activity, to be preferably used as a neo-adjuvant therapy, which does not have the typical side effects of all anti-neoplastic agents, including hydroxyurea.

Such an object is accomplished by the present inventors, who have found that albumin exerts an antiproliferative and differentiating effect and is therefore effective as an active ingredient for treating cancer. The inventors have also found that albumin, at suitable concentrations and in combination with conventional anti-neoplastic agents, increases considerably the effectiveness of the anti-neoplastic agent, allowing the active principle dose to be reduced, therefore causing a reduction of the most common side effects.

Anti-cancer medicaments including albumin as a stabilizing agent or as a carrier are known from the prior art, including albumin-anticancer drug conjugates which are capable of enhancing drug incorporation by tumor cells. However, albumin had never been disclosed as an active ingredient per se in an anti-cancer medicament.

The effectiveness of albumin as an active ingredient in an anti-cancer medicament is unexpected over the prior art.

For example in [4] there is disclosed that the cytotoxicity against tumor cells (human CCRF-CEM leukaemia and GC3 colon carcinoma cells) of the anti-neoplastic agent diaryl-sulfonylurea (DSU) is markedly enhanced when a serum- and albumin-free medium is used, in comparison with a normal growth medium such as a RPMI-1640 containing 10% FCS.

A first subject-matter of the invention is therefore albumin as an active ingredient in a medicament for the treatment of cancer.

A second subject-matter of the invention is a pharmaceutical composition comprising a pharmaceutically effective amount of at least one anti-neoplastic agent and albumin, in a pharmaceutically acceptable vehicle. A pharmaceutically effective amount of albumin as an anti-neoplastic active ingredient (that is to say, anti-proliferative and pro-differentiating) is at least 2% w/v of the composition.

Preferably, the anti-neoplastic agent used in the pharmaceutical composition of the invention is an anti-proliferative agent, such as hydroxyurea.

However, albumin has been found to effectively reduce the dose, and consequently the side effects, of other anti-neoplastic agents as well. Consequently, any other anti-neoplastic agent may be comprised in the composition of the invention in replacement of hydroxyurea, such as for example cyclophosphamide, iphosphamide, lomustine, carmustine, streptozotocin, fotemustin, temozolomide, dacarbazine, metotrexate, fluorouracil, gemcitabine, vinblastine, vincristine, vinorelbine, doxorubicin, epirubicin, mitoxantron, bleomycin, mitomycin, procarbazine, megestrol, medroxyprogesterone, leuprorelin, triptorelin, tamoxifen, letrozole, BCG vaccine, cyclosporin, azathioprine, analogues, precursors and derivatives thereof.

Preferably, the concentration of hydroxyurea in the pharmaceutical composition of the invention is between 10 and 1 mM, more preferably between 10 μM and 100 μM.

The pharmaceutical composition of the invention is suitable to be administered as a cancer therapy to any mammal, including human beings. In a preferred embodiment, the albumin used in the pharmaceutical composition is an albumin specific for the species to which the subject to be treated belongs.

The pharmaceutical composition of the invention preferably is a combination of an anti-neoplastic agent and albumin in physiologic solution. As indicated above, a pharmaceutically effective concentration of albumin is at least 2% (w/v). The preferred albumin concentration is between 2% and 50% (w/v), more preferably 10-20% (w/v).

The pharmaceutical composition of the invention can be administered for example by injection, by infusion directly into those arteries that supply the area connected to a tumour, or by intra-tumour infusion, reducing the tumour mass in a short temporal range with a daily administration regime. Other administration routes can be contemplated, such as for instance the intravenous, oral or topical ones.

The administration dose is determined according to various factors, such as the species to which the subject to be treated belongs, the subject's body weight, the morphology, hysto-type, size, and classification of the tumour to be treated, and can be determined by a person of ordinary skill in the art by using his/her normal knowledge.

The combination of the anti-neoplastic agent and albumin is preferably used as a neo-adjuvant anti-neoplastic agent. Neo-adjuvant, or primary, therapy means the administration of anti-proliferative drugs which precedes the locoregional therapy (surgery and/or radio-therapy) in patients presenting bulky or locally advanced tumours. The goals of the pre-surgical or pre-radiotherapeutic pharmacological treatment are (i) making a locally advanced neoplasia radically operable; (ii) allowing for the execution of a conservative surgical operation, if the size of the neoplasia requires a radical surgery; (iii) monitoring in vivo the responsiveness to the treatments; (iv) precocious administration of the anti-proliferative drugs in order to eradicate the micro-metastases responsible for relapse of the disease.

Many studies have proved that it is possible to reduce the size of the neoplasia by pre-operatively administering some cycles of chemotherapy, and recent data also indicate the effectiveness of endocrinal therapies for such an aim in patients with positive hormone receptors [5]. Such a reduction in the tumour size makes a radical surgery operation possible in patients with a locally advanced disease. Furthermore, the original tumour being reduced in size by the primary systemic therapy allows for a conservative surgery (quadrantectomy) in neoplasias that could be operated on but that due to their initial size should have been subjected to mastectomy. A further objective of the primary systemic treatment would be to interfere precociously with the proliferation of possible micro-metastases already present at diagnosis. Despite the high percentage of therapeutic responses that made it possible to perform a conservative surgery in many women, the disease-free and overall survival rate has not changed compared to standard treatment (surgery followed by adjuvant chemotherapy). The primary systemic treatment should be reserved for patients with a true possibility of benefiting from the reduction of the primary tumour size and, possibly, of the extent of the homolateral metastases. With regard to the chemotherapy to be used, any type of regime can be applied, provided that it has proven effective in the therapy for the advanced disease [6].

The inventors have found that, for the combination of the anti-neoplastic agent and albumin of the invention to be effective, for example in the anti-proliferative therapy described above, the two components do not need to be administered as a physical mixture thereof. The two components can be administered simultaneously, separately, or sequentially, in any order.

Therefore, another object of the invention is a kit comprising at least one anti-neoplastic agent and albumin as a combined preparation for simultaneous, separate or sequential use in cancer therapy.

Without wishing to be bound by any theory, the inventors believe that the increased effectiveness of the anti-neoplastic therapy seen with the combination of the anti-neoplastic agent and albumin may be linked to the function of albumin as a selective amino acid carrier. In other words, albumin has the effect of providing a nourishment for cells with the same amino acids that compose it, in a way that is proportional to such a composition, inducing a pro-differentiating mitochondrial oxidative metabolism. Albumin is in fact catabased by all extra-hepatic tissues and its leakage from the intravascular compartment to the extra-vascular one is favoured by small damages in the vessel walls, by hypoxia and insulin. Postprandial hyperinsulinaemia, by stimulating the synthesis of albumin, facilitates a temporary deposit of the essential amino acids ingested with food into the protein. Once broken down, albumin places the amino acids that compose it, ingested about 20 days earlier, at the disposal of muscle and other tissues.

Ageing is connected with a reduction in lean mass. Albuminaemia is lower in the elderly and is positively correlated with the reduction in muscle mass. This finding suggests a role played by albumin:
in favour of the muscle anabolism
that is nutritional and antioxidant in that, during absorption of the meal, its synthesis rate increases by 50%, favouring the incorporation of a considerable amount of essential amino acids ingested with food and avoiding the irreversible oxidation thereof;
the effect of providing a nourishment for cells with the same amino acids that compose it, in a way that is proportional to such a composition, and thus selective, inducing a pro-differentiating mitochondrial oxidative metabolism.

The amino acid sequence of human albumin (HAS) is known per se. The mature protein, having a molecular weight of 66 kDa is 585 amino acids in length.

Human albumin is a normal constituent of human plasma and has the same activity as physiologic albumin. Checking toxicity after a single administration has little clinical significance and does not make it possible to evaluate the toxic or lethal dose nor to define a dose/effect relationship. Under normal conditions, the half-life of albumin is on average 19 days and its concentration is 4-5 g/kg body weight, 40-45% of which is in the intravascular space and 55-60% in the extra-vascular one. But in certain cases, there may be an abnormal distribution, such as for instance within the first 24 hours after serious bums and during septic shock. Under normal conditions, the half-life of albumin is on average 19 days. The balance between synthesis and catabolism occurs according to a feedback regulation. The clearance mostly takes place in the intracellular space by lysosomal proteases. Less than 10% of the infused albumin leaves the intravascular compartment during the first two hours after the infusion; as a result, the circulating volume increases from the 1^{st} to the 3^{rd} hour after the administration. When human albumin is used in the restoration therapy, the required dosage is suggested by common circulating parameters. To date, no human albumin-associated oncogenic or mutagenic embryofetotoxic potentials have been reported. No signs of acute toxicity are described in animal models.

Several bio-active substances, drugs or vaccines can be bound and combined with albumin for human, veterinary or agricultural use. Albumin induces stabilization of drug and vaccine activities. When combined or bound with albumin, some drugs also exert their effect at lower concentrations compared to the formulation without albumin. The extent of the distribution of drugs in tissues depends on the extent of the bond with the plasma proteins (e.g. acidic drugs generally tend to bind primarily with albumin) and with the tissues themselves.

In order to prove the effectiveness of the combination of the invention of an anti-neoplastic agent and albumin, the inventors carried out experiments both in vitro and in vivo.

Experiments performed in vitro on different adherent neoplastic cell types (such as Hep-G2 hepatocarcinoma cell lines, primary canine hepatocarcinoma cells, primary human mammary tumour cells, primary canine mammary tumour cells, human glioblastoma cells) showed that use of albumin dissolved in physiologic solution at a concentration, expressed in weight to final volume, within a range from 2% to 50%, and preferably from 10% to 20%, can cause about 50-100% of the cultured cells to die within 8-24 hours of incubation.

Experiments performed in vitro on different neoplastic cell types, both in suspension and adherent (in suspension: lymphoblastic leukaemia cell lines such as H9, MT2, MT4, THP-1 and U937 monocytic leukaemia cell lines, K562 erythroid leukaemia cell lines; adherent: human Hep-G2 hepatocarcinoma cell lines, primary canine hepatocarcinoma cells, primary human mammary tumour cells, primary canine mammary tumour cells, human glioblastoma cells) also showed that use of albumin in physiologic solution at a concentration, expressed in weight to final volume, within a range from 2% to 50% w/v, preferably within a range from 10% to 20% w/v together with hydroxyurea at a final concentration preferably within a range of 10-100 microMolar, can cause about 80-100% of the cultured cells to die within 4-12 hours of incubation.

Materials and Methods

For the in vitro and in vivo experiments, the pharmaceutical composition of the invention was prepared by using species-specific albumin and hydroxyurea, in the quantities indicated in Tables 2 and 3, respectively. For all the formulations listed below, the substances were weighed as required by the formula, obtaining one final litre of solution.

TABLE 2
Culture medium: in vitro use

| Substance | Concentration/L | |
|---|---|---|
| ALBUMIN | 100 | grams |
| HYDROXYUREA | 100 | μM |
| physiologic solution | q.s. to 1 | litre |

TABLE 3
Pharmaceutical composition: in vivo use

| Substance | Concentration/L | |
|---|---|---|
| ALBUMIN | 100 | grams |
| HYDROXYUREA | 1 | mM |
| physiologic solution | q.s. to 1 | litre |

Pharmaceutical Composition for in vivo Use: Intervention Protocol.

In order to avoid eventual anaphylactic shock events after the intra-articular infusion therapy, a prophylaxis can be performed prior to the administration with antihistamines and/or cortisones, such as, for example, Diphenylhydramine, histamine receptor type 1 antagonists, Cetirizine, Loratidine, Fexophenadine, Betamethasone disodium phosphate, Hydrocortisone, Methylprednisolone.

Cell Lines

The following cells were used for setting up the experiments in vitro:

in suspension: lymphoblastic leukaemia cell lines such as H9, MT2, MT4, THP-1 and U937 monocytic leukaemia cell lines, K562 erythroid leukaemia cell lines;

adherent: Hep-G2 hepatocarcinoma cell lines, primary canine hepatocarcinoma cells, primary human mammary tumour cells, primary canine mammary tumour cells, human glioblastoma cells.

The cells were washed three times by centrifuging at 160 g for 10 min at room temperature in phosphate buffer (PBS, pH 7.2) and resuspended into 50 ml tubes (Nunc, Kamstrup, Denmark) at a final concentration of 10×10⁶ cells/ml, again in phosphate buffer (PBS, pH 7.2).

The cells were resuspended at a final concentration of 1×10⁶ cells/ml into six-well plates (Lab-Tek chamber slides, Nunc, Kamstrup, Denmark) in 4 ml/well of final solution consisting of common RPMI 1640 culture medium (GIBCO) supplemented with:
10% FCS (Celbio, Milan, Italy);
100 units/ml penicillin;
100 μg/ml streptomycin;
160 mg/L gentamycin (Schering-Plough, Milan, Italy);
2 mM L-glutamine (Life Technologies; growth medium).

The cells were incubated for 3 days in a thermostatically controlled Heraeus incubator at a temperature of 37° C. under an atmosphere having a constant supply of 5% CO₂ (v/v in air).

After incubation, the cells were all eutrophic with a specific morphology for each cell type.

Each six-well plate is split as follows:
CONTROL (negative control) two wells had no treatment and the cells were cultured in the culture medium described above;
SAMPLE-1 (sample treated with 20% ALBUMIN alone) two wells were assigned for treatment with albumin alone at 20% concentration expressed as weight/final volume of culture medium as described above;
SAMPLE-2 (sample treated with the combination ALBUMIN/HYDROXYUREA, designated as BIN-ALB) two wells were assigned for treatment with 10% albumin and 50 micromolar HYDROXYUREA, the concentrations being expressed as weight/final volume of culture medium as described above.

The cells were monitored hourly for a total of three days of culture. By the third day of incubation, all of the cultured cells had constantly been shown to die.

Trypan Blue Staining.

Trypan blue is a dye capable of selectively staining dead cells. The reason why this dye does non stain living cells is due to the extreme selectivity of the cell membrane. Living cells, by having an intact membrane, do not allow this dye to penetrate the cytoplasm; on the contrary, it easily penetrates dead cells, making them distinguishable from the living cells by a rapid microscopic analysis. Trypan blue is not able to distinguish between apoptotic cells and necrotic cells. The cell suspensions are incubated with 5% trypan blue for 5 minutes at room temperature; once the incubation is completed, 10 microlitres of this stained cell solution are taken and deposited into a cell counting chamber (e.g. hemocytometer chamber) and a careful examination is performed with a light microscope at 20× and 40× sequential magnitudes. Thus, one proceeds with the counting of the number of living and dead cells per ml. Dead cells will appear intensely coloured in blue, the living cells will not be coloured in blue.

MTT Staining

This assay estimates the viability of cells cultured under certain conditions. In fact, living cells are able to chemically break down the compound MTT, a water-soluble tetrazolium salt, which becomes a violet-coloured and water-insoluble formazan salt [8]. The treatment of the cells is performed in a NUNC 96-well plate, assigning 6 wells for each experimental condition, at a density of 15000 cells per well, in a final volume of 200 microlitres per well containing MTT at a final concentration of 0.5 mg/ml. The plate is placed into a thermostatically controlled Heraeus incubator at a temperature of 37° C. under an atmosphere having a constant supply of 5% CO₂ (v/v in air) for 30 minutes. Once the incubation is completed, the medium is removed from the wells by gentle suction with a multichannel pipette. After having ascertained that no traces of solution are still in the wells, 100 ml of DMSO are added to each well and the plate is shaken for 10 minutes at room temperature so as to melt the formazan salt crystals. If the cells are metabolically viable, therefore reducing MTT, when DMSO is added, the development of a violet colour can be observed. The viability is quantified by reading the absorbance with a spectrophotometer reader for ELISA plates, at a 570 nm wavelength. Each experiment was repeated at least 3 times and the data have been expressed as the percentage of the control, wherein the ability to reduce MTT was taken as 100% viability.

Hematoxylin-Eosin Staining.

This is the basic staining in the microscopic analysis of animal tissues and it enables a better morphological study thereof under a light microscope. Through hematoxylin or Mayer's emallume, it colours in blue the negatively charged cell components, such as nucleic acids, membrane and cell proteins, elastin, which are therefore designated as basophils. Through eosin, it colours in red the positively charged (acidic) components, such as cell proteins in certain cells (eosinophils) and collagen fibres, which are therefore designated as acidophils.

Useful Material
a slide with an unstained section
a coverslip
a slide support having a handle at least 5-10 cm long
10 glass tanks of a size compatible with that of the slide support
   7 of which will constitute the hydration/dehydration sequence
   one is used for hematoxylin
   one for eosin
   one for the washes
xylene, 2 tanks
absolute ethanol, 2 tanks
ethanol 90°, 2 tanks
Mayer's emallume (Hematoxylin)
Alcoholic eosin solution
distilled water, 1 litre

Hydration

Place the slide into the 60° C. oven until the paraffin becomes liquid. Thereafter the slide is dipped:
1. 20 minutes into xylene 1
2. 15 min into xylene 2
3. 10 min into absolute ethanol 1
4. 10 min into absolute ethanol 2
5. 5 min into ethanol 90° 1
6. 5 min into ethanol 90° 2
7. 20 min into distilled water

Hematoxylin
1. dip the slides into the dye for 20 seconds
2. wash twice in distilled water
3. place the tank with the slides under running tap water for 5 min, if possible without aiming the flow directly at the sections
4. wash in distilled water

Eosin
1. dip the slide support into the tank containing the alcohol 90° for 15 min
2. dip the slides into eosin for 1 minute
3. then place them into the washing tank filled with alcohol 90°

Dehydration
1. Proceed with the alcohols and the xylene, and finally lay a drop of mounting balsam and place the coverslip.

### EXAMPLE 1

### Light Microscopy

At 12, 24, 48, and 72 hours of incubation, the samples showed, with hematoxylin-eosin staining, an increasing cell death up to 100% of cultured cells for all of the tested cell types after the 72^{nd} hour.

The controls exhibited a widespread eutrophism with a characteristic morphology and a 90%-100% viability for all of the cell types tested.

The results pointed out that the use of albumin dissolved in physiologic solution at a concentration, expressed as weight to final volume, within a range from 2% to 50% and preferably from 10% to 20% can cause 50%400% of all the cultured cells to die within 8-24 hours of incubation.

Moreover, the results pointed out that the use of albumin in physiologic solution at a concentration, expressed as weight to final volume, within a range from 10% to 20% together with hydroxyurea at a final concentration within a range of 10-100 microMolar can cause 80%-100% of all the cultured cells to die within 4-12 hours of incubation.

### EXAMPLE 2

### Quantification by Trypan Blue of the Cell Viability Between Treated Samples versus Untreated Samples

The results related to viability and mortality in samples and relevant controls after a 24 hour incubation have been expressed as a scale of percentage values, as follows in:
Table 4 (that is, treatment with ALBUMIN at 20% (w/v) of final solution),
Table 5 (treatment with the compound 10% ALBUMIN and 100 μM HYDROXYUREA, expressed as weight/volume in the final solution).

TABLE 4

| CELLS treated with 20% Albumin | VIABILITY | MORTALITY |
|---|---|---|
| H9 cell line | 10% | 90% |
| Untreated control | 99% | 1% |
| MT2 cell line | 15% | 85% |
| Untreated control | 97% | 3% |
| MT4 cell line | 19% | 81% |
| Untreated control | 97% | 3% |
| THP-1 cell line | 18% | 82% |
| Untreated control | 98% | 2% |
| U937 cell line | 14% | 86% |
| Untreated control | 98% | 2% |
| K562 cell line | 19% | 81% |
| Untreated control | 91% | 9% |
| HEP-G2 cell line | 20% | 80% |
| Untreated control | 89% | 11% |
| Primary canine hepatocarcinoma cells | 28% | 72% |
| Untreated control | 88% | 12% |
| Primary human mammary tumour cells | 12% | 88% |
| Untreated control | 88% | 12% |
| Primary canine mammary tumour cells | 30% | 70% |
| Untreated control | 91% | 9% |
| Human glioblastoma cells | 11% | 89% |
| Untreated control | 98% | 2% |

TABLE 5

| CELLS treated with Albumin/ | | |
|---|---|---|
| Hydroxyurea | VIABILITY | MORTALITY |
| H9 cell line | 5% | 95% |
| Untreated control | 99% | 1% |
| MT2 cell line | 8% | 92% |
| Untreated control | 97% | 3% |
| MT4 cell line | 5% | 95% |
| Untreated control | 97% | 3% |
| THP-1 cell line | 1% | 99% |
| Untreated control | 98% | 2% |
| U937 cell line | 4% | 96% |
| Untreated control | 98% | 2% |
| K562 cell line | 7% | 93% |
| Untreated control | 91% | 9% |
| HEP-G2 cell line | 10% | 90% |
| Untreated control | 89% | 11% |
| Primary canine hepatocarcinoma cells | 10% | 90% |
| Untreated control | 88% | 12% |
| Primary human mammary tumour cells | 8% | 92% |
| Untreated control | 88% | 12% |
| Primary canine mammary tumour cells | 10% | 90% |
| Untreated control | 91% | 9% |
| Human glioblastoma cells | 3% | 97% |
| Untreated control | 98% | 2% |

### EXAMPLE 3

### Quantification by MTT of the Cell Viability Between Treated Samples Versus Untreated Samples

The results related to viability and mortality in samples and relevant controls after a 48 hour incubation have been expressed as a scale of percentage values compared to the control wherein the ability to reduce MTT was taken as 100% viability. The results are illustrated in Table 6.

TABLE 6

| CELLS treated with Albumin/ | | |
|---|---|---|
| Hydroxyurea | VIABILITY | MORTALITY |
| Untreated control | 100% | 0% |
| H9 cell line | 3% | 97% |
| MT2 cell line | 4% | 96% |
| MT4 cell line | 3% | 97% |
| THP-1 cell line | 1% | 99% |
| U937 cell line | 3% | 97% |
| K562 cell line | 2% | 98% |
| HEP-G2 cell line | 2% | 98% |
| Primary canine hepatocarcinoma cells | 3% | 97% |
| Primary human mammary tumour cells | 3% | 97% |
| Primary canine mammary tumour cells | 5% | 95% |
| Human glioblastoma cells | 3% | 97% |

### EXAMPLE 4

### BIN-ALB-INFUS Pharmaceutical Composition. Clinical Study in Dogs

Protocol

1. Recruitment

Ten dogs (canid species of different breed, size, body weight, sex and age).

2. Inclusion Criteria

Solid tumours (Mammary Carcinoma, etc.) with a lesion to be treated not bigger than 3 cubic cm.

3. Clinical Procedure

The following procedures have been performed in all of the dogs tested.

a. Time Point Zero
Collection of medical history data and clinical examination comprising complete hematochemical evaluation+manual count of the leukocyte formula (assessment of possible leukopaenia), EPP (electrophoretic protein pattern), albuminaemia, liver and pancreas enzymes, CPK, LDH and the LACTATES for assessment of the metabolic oxidation;
Ultrasound scan of the lesion concerned;
Histological examination of a sample;
Tumour staging (TNM);
Evaluation of locoregional phlebitis and thrombo-phlebitis, description in medical history file (prognostic significance as to effectiveness of the treatment);
Evaluation of both multifocal lesions and locoregional stations, description in medical history file (prognostic significance as to effectiveness of the treatment);
Scan-guided intra-lesion inoculation (ideally into the centre(s) of the lesion(s)) of 1 mL of tested solution per cubic cm of the lesion diameter.

The clinical examination with locoregional evaluation and the ultrasound scan of the lesion, with repetition of the scan-guided intra-lesion injection of the solution, were performed weekly and four/eight times, depending on the seriousness of the initial pathological condition.

b. End of the Treatment Course
Ultrasound scan and assessment of the size of the treated lesion;
Histological examination of a sample;
Tumour staging (TNM);
Evaluation of locoregional phlebitis and thrombo-phlebitis, description in medical history file (prognostic significance as to effectiveness of the treatment);
Evaluation of both multifocal lesions and locoregional stations, description in medical history file (prognostic significance as to effectiveness of the treatment);
Clinical examination with medical reporting, comprising complete hematochemical evaluation+manual count of the leukocyte formula (assessment of possible leukopaenia), EPP (electrophoretic protein pattern), albuminaemia, liver and pancreas enzymes, CPK, LDH and the LACTATES for assessment of the metabolic oxidation.
Conclusions with regard to EFFECTIVENESS and TOXICITY.

Notes

The laboratory used is recognized by Regione Piemonte under AUT.G.R. as from 10/12/1990 N. 193-2412. The case record was collected by the Associate Veterinary Surgery, corso Traiano 99/d TO (HEALTH DIR. doctor C. Vercelli) as the Reference Centre and by any Associate Veterinary Surgery connected with the Reference Centre.

Conclusions

The use of the combination of albumin and hydroxyurea in the clinical studies performed on 10 dogs of different breed and size proved to be excellently tolerable in all of the subjects without showing any apparent systemic-type effect, irrespective of the different age, size, sex and localisation of the tumour pathology. The EFFECTIVENESS of the treatment resulted in the remarkable reduction of the tumour mass prior to the surgical removal.

### REFERENCES

1. Styles L A, Lubin B, Vichinsky E, Lawrence S, Hua M, Test S, Kuypers F. Decrease of very late activation antigen-4 and CD36 on reticulocytes in sickle cell patients treated with hydroxyurea Blood. 1997 Apr. 1; 89(7):2554-9.
2. Burnett A K, Milligan D, Prentice A G, Goldstone A H, McMullin M F, Hills R K, Wheatley K. A comparison of low-dose cytarabine and hydroxyurea with or without all-trans retinoic acid for acute myeloid leukemia and high-risk myelodysplastic syndrome in patients not considered fit for intensive treatment. Cancer. 2007 Mar. 15; 109(6):1114-24.
3. Ballas S K, Barton F B, Waclawiw M A, Swerdlow P, Eckman J R, Pegelow C H, Koshy, M, Barton B A, Bonds D R. Hydroxyurea and sickle cell anemia: effect on quality of life. Health Qual Life Outcomes. 2006 Aug. 31; 4:59.
4. Schultz R M et al. Effect of albumin on antitumor activity of diarylsulfonylureas. Anticancer Res. 1993 November-December; 13(6A): 1939-43.
5. Ebara S, Manabe D, Kobayashi Y, Tanimoto R, Saika T, Nasu Y, Saito S, Satoh T, Miki K, Hashine K, Kumon H. The efficacy of neoadjuvant androgen deprivation therapy as a prostate volume reduction before brachytherapy for clinically localized prostate cancer. Acta Med Okayama. 2007 December; 61(6):335-40.
6. Rustogi A, Budrukkar A, Dinshaw K, Jalali R. Management of locally advanced breast cancer: evolution and current practice. J Cancer Res Ther. 2005 January-March; 1(1):21-30. Review.
7. Castro-Garza J, Barrios-García H B, Cruz-Vega D E, Said-Fernández S, Carranza-Rosales P, Molina-Torres C A, Vera-Cabrera L. Use of a colorimetric assay to measure differences in cytotoxicity of Mycobacterium tuberculosis strains. J Med Microbiol. 2007 June; 56(Pt 6):733-7.

1.-17. (canceled)

18. A medicament for treating cancer, the medicament comprising albumin as an active ingredient.

19. The medicament of claim 18, wherein the albumin is comprised in combination with an anti-neoplastic agent.

20. The medicament of claim 19, wherein the anti-neoplastic agent is an anti-proliferative agent.

21. The medicament of claim 20, wherein the anti-proliferative agent is selected from the group consisting of hydroxyurea such as for example cyclophosphamide, iphosphamide, lomustine, carmustine, streptozotocin, fotemustin, temozolomide, dacarbazine, metotrexate, fluorouracil, gemcitabine, vinblastine, vincristine, vinorelbine, doxorubicin, epirubicin, mitoxantron, bleomycin, mitomycin, procarbazine, megestrol, medroxyprogesterone, leuprorelin, triptorelin, tamoxifen, letrozole, BCG vaccine, cyclosporin, azathioprine, analogues, precursors and derivatives thereof.

22. The medicament of claim 21, wherein the anti-proliferative agent is hydroxyurea at concentration between 10 μM and 1 mM.

23. The medicament of claim 21, wherein the anti-proliferative agent is hydroxyurea at concentration between 10 μM and 100 μM.

24. The medicament of claim 18, wherein the albumin is comprised at a concentration between 2 and 50% w/v.

25. The medicament of claim 18, wherein the albumin is comprised at a concentration between 10 and 20% w/v.

26. The medicament of claim 18, wherein the medicament is formulated for administration to a species and wherein the albumin is specific for said species.

27. The medicament of claim 18, wherein the medicament is formulated for administration by infusional, injective, oral or topical route.

28. The medicament of claim 18, wherein the medicament is formulated for a neo-adjuvant anti-proliferative therapy.

29. A pharmaceutical composition comprising at least one anti-neoplastic agent and albumin in a pharmaceutically acceptable vehicle, wherein the albumin is comprised at a concentration of at least 2% w/v of the composition.

30. The pharmaceutical composition of claim 29, wherein the albumin is comprised at a concentration between 2 and 50% w/v of the composition.

31. The pharmaceutical composition of claim 30, wherein the albumin is comprised at a concentration between 10 and 20% w/v of the composition.

32. The pharmaceutical composition of claims 29, wherein the anti-neoplastic agent is an anti-proliferative agent.

33. The pharmaceutical composition of claims 32, wherein the anti-proliferative agent is selected from the group consisting of hydroxyurea such as for example cyclophosphamide, iphosphamide, lomustine, carmustine, streptozotocin, fotemustin, temozolomide, dacarbazine, metotrexate, fluorouracil, gemcitabine, vinblastine, vincristine, vinorelbine, doxorubicin, epirubicin, mitoxantron, bleomycin, mitomycin, procarbazine, megestrol, medroxyprogesterone, leuprorelin, triptorelin, tamoxifen, letrozole, BCG vaccine, cyclosporin, azathioprine, analogues, precursors and derivatives thereof.

34. The pharmaceutical composition of claim 32, wherein the anti-proliferative agent is hydroxyurea at concentration between 10 μM and 1 mM

35. The pharmaceutical composition of claim 29, wherein the pharmaceutical composition is formulated for administration to a species and wherein the albumin is specific for said species.

36. The pharmaceutical composition of claim 29, wherein the medicament is formulated for administration by infusional, injective, oral or topical route.

37. A kit of parts, comprising at least one anti-neoplastic agent and albumin as a combined preparation for simultaneous, separate or sequential use in the treatment of cancer.

38. A method for treating cancer in a subject, the method comprising
administering to the subject an effective amount of albumin alone or in combination with an anti-neoplastic agent.

39. The method of claim 38, wherein the anti-neoplastic agent is an anti-proliferative agent.

40. The method of claim 39, wherein the anti-proliferative agent is selected from the group consisting of hydroxyurea such as for example cyclophosphamide, iphosphamide, lomustine, carmustine, streptozotocin, fotemustin, temozolomide, dacarbazine, metotrexate, fluorouracil, gemcitabine, vinblastine, vincristine, vinorelbine, doxorubicin, epirubicin, mitoxantron, bleomycin, mitomycin, procarbazine, megestrol, medroxyprogesterone, leuprorelin, triptorelin, tamoxifen, letrozole, BCG vaccine, cyclosporin, azathioprine, analogues, precursors and derivatives thereof.

41. The method of claim 38, wherein the administering is performed by infusional, injective, oral or topical route.

42. The method of claim 38, wherein the administering is performed in a neo-adjuvant anti-proliferative therapy.
